(19) United States
(12) Patent Application Publication
(10) Pub. No.: US 2014/0358226 A1
(43) Pub. Date: Dec. 4, 2014

(54) Light Adjustable IOL With Diffraction Multifocal

(71) Applicant: Allen Louis Cohen, Manakin Sabot, VA (US)

(72) Inventor: Allen Louis Cohen, Manakin Sabot, VA (US)

(21) Appl. No.: 14/460,386

(22) Filed: Aug. 15, 2014

(51) Int. Cl. A61F 2/16

(52) U.S. Cl. CPC A61F 2/1654; A61F 2210/00
USPC 623/6.31

(57) ABSTRACT

A light adjustable diffraction multifocal intraocular lens (IOL), according to the present invention, is a mutifocal IOL whose optical power (prescription) may be adjusted by exposure to an energy source after implantation and subsequent wound healing. It makes use of a diffraction element to create the multifocal nature of the IOL which retains its structural integrity despite exposure to the power changing energy source.

### BACKGROUND OF THE INVENTION

1. Field of the Invention

Many people, particularly the aged, develop cataracts necessitating the removal of the natural crystalline lens from within their eye. Intraocular lenses (IOLs) are prosthetic lenses that are used as a replacement for the natural lens of the human eye. The present invention relates generally to IOLs, and more particularly to light adjustable diffraction bifocal IOLs.

Multifocal IOLs have two or more distinct powers that provide a patient, who has undergone cataract surgery, with at least far and near vision prescriptions. There exist both refractive multifocal IOLs (ReZOOM®, for instance) and diffraction multifocal IOLs (Tecnis® multifocal, ReSTOR® and OptiVis®, for instance). But, diffraction multifocal IOLs are known to provide better visual quality and have a greater patient success rate than refractive multifocal IOLs.

Light adjustable IOLs have the property of allowing the IOL prescription power to be non-invasively modified after implantation within the eye. That is, any errors in the pre-surgical power calculation due to imperfect measurements and/or variable lens positioning and wound healing may be modified in a simple post-surgical outpatient procedure.

The present invention, a light adjustable diffraction multifocal IOL would provide both the sharp acuity of a diffraction multifocal IOL and also be able to have it's prescription easily modified after implantation within the eye.

2. Description of the Prior Art

### a. Diffraction Multifocal IOL

A diffraction lens generally comprises a number of concentric annular zones of equal area with optical steps separating the adjacent annular zones. The width of the zones determines the separation between the diffraction powers (i.e. the far and near vision prescriptions) of the lens. The separation of the diffraction powers increases with decreasing zone width and vice versa. The depth of the optical steps determines the intensity split of light between the two prescriptions. With deeper optical steps, more light is directed to the near prescription, while with shallower optical steps, more light is directed to the far prescription, thereby providing improved contrast at the near or far focus respectively.

Beginning in the 1980s Cohen (U.S. Pat. Nos. 4,210,391; 4,338,005; 4,340,283; 4,881,804; 4,881,805; 5,056,908; 5,120,120; 5,121,979; 5,121,980; 5,144,483) and Freeman (U.S. Pat. Nos. 4,637,697; 4,642,112; 4,655,565; 4,641,934) disclosed the first ophthalmic diffraction multifocal lenses with blazed (angled) zones exhibiting a saw-toothed profile. These blazed lens designs, for the most part, had substantially parabolic profiles with ½ wave deep optical steps. This design profile produces bifocal lenses that divide light equally between the 0th (far vision) and +1 (near vision) diffraction orders.

In the 1990s Cohen (U.S. Pat. Nos. 4,995,714; 4,995,715; 5,054,905) disclosed the first ophthalmic diffraction multifocal lenses with zone profiles exhibiting otherwise varied curvatures and shapes. In particular, these lens designs used non-parabolic profiles with optical steps which were either more or less than ½ wave deep and still divided light equally between the 0th and +1 diffraction orders.

### b. Light Adjustable IOL

In the early 2000s, Jethmalani, et al. (U.S. Pat. Nos. 6,450,642; 6,813,097; 6,824,266 and 6,851,804); Sandstedt et al. (U.S. Pat. No 6,749,632); and Chang, et al. (U.S. Pat. Nos. 7,074,840 and 7,237,893) disclosed the first light adjustable IOLs wherein the prescription of the IOL lens body was directly altered in response to exposure to a laser light beam. These were single vision lenses that had the property of having their corrective power altered by a simple post-surgical outpatient procedure in which the IOLs were exposed to a focused laser light beam.

### c. Light Adjustable Multifocal IOL

Shortly thereafter, Sandstedt, et al. (U.S. Pat. Nos. 7,281,795 and 7,988,285; U.S. Application Nos. 20030151831 A1 and 20050099597 A1) disclosed the first light adjustable multifocal IOLs. These were multifocal IOLs that could have their corrective power altered by a simple post-surgical outpatient procedure in which the IOLs were exposed to a focused laser light beam.

However, the multifocality of these prior art lenses is inherently refractive in nature. In fact, the disclosures by Sandstedt, et al., suggest creating the multifocality of light adjustable multifocal IOLs by exposing alternating concentric zones to laser light of a varying spatial intensity profile so that “physical changes in the radius of curvature of the lens surface are achieved, thus modifying the refractive power of the lens.” Such power changes created by altering the radius of curvature of a lens surface are the hallmark of refractive lens design. By contrast, the multifocality of a diffraction lens design does not depend up on the radii of curvature of a lens surface, but depends only on the zone widths and step heights of a diffraction profile.

Moreover, since the mechanism of altering the corrective power of a light adjustable IOL post-surgically involves a differential swelling of the IOL lens body, any diffraction profile incorporated into these lenses would be destroyed by such a post-surgical procedure. This is so, because the zone widths and step heights of a diffraction profile, must be maintained with very precise tolerances that would be disturbed by any differential swelling of the IOL lens body.

### BRIEF SUMMARY OF THE INVENTION

Prior art light adjustable multifocal IOLs, are designed according to the principles of refractive optics. They utilize annular zones of varying radii of curvature in order to manifest multifocality. This invention relates to light adjustable multifocal IOLs that utilize diffraction lens designs comprising annular zones separated by optical steps.

It is an object of the present invention to provide a light adjustable multifocal IOL that would provide both the sharp acuity of a diffraction multifocal IOL and also be able to have it's prescription easily modified after implantation within the eye.

It is a further object of the present invention to provide a light adjustable diffraction multifocal IOL wherein the diffraction profile is not disturbed by any differential swelling of the IOL lens body that occurs when the corrective power of the IOL is post-surgically altered by irradiation with a laser light beam.

### BRIEF DESCRIPTION OF THE DRAWINGS

FIG. 1a is a cross-section view of a multifocal intraocular lens of this invention.

FIG. 1b is a cross-section view of another embodiment of the multifocal intraocular lens of this invention.

FIG. 1c is a plan view of the posterior surface of multifocal intraocular lens of this invention showing the concentric annular zones on the diffraction profile.

FIG. 2 is a schematic representation of one embodiment of the diffraction profile of the lens of the present invention.

FIG. 3 illustrates a post-surgical procedure for inducing a power change in a prior art light adjustable IOL by altering the refractive index of the IOL.

FIG. 4 illustrates a post-surgical procedure for inducing a power change in a prior art light adjustable IOL by altering the radius of curvature of the IOL.

FIG. 5 illustrates a post-surgical procedure for inducing multifocality in a prior art light adjustable IOL by altering the radius of curvature of an annular region of the IOL.

FIG. 6 illustrates a post-surgical procedure for inducing a power change in the light adjustable diffraction multifocal IOL of the present invention.

### DETAILED DESCRIPTION OF THE INVENTION

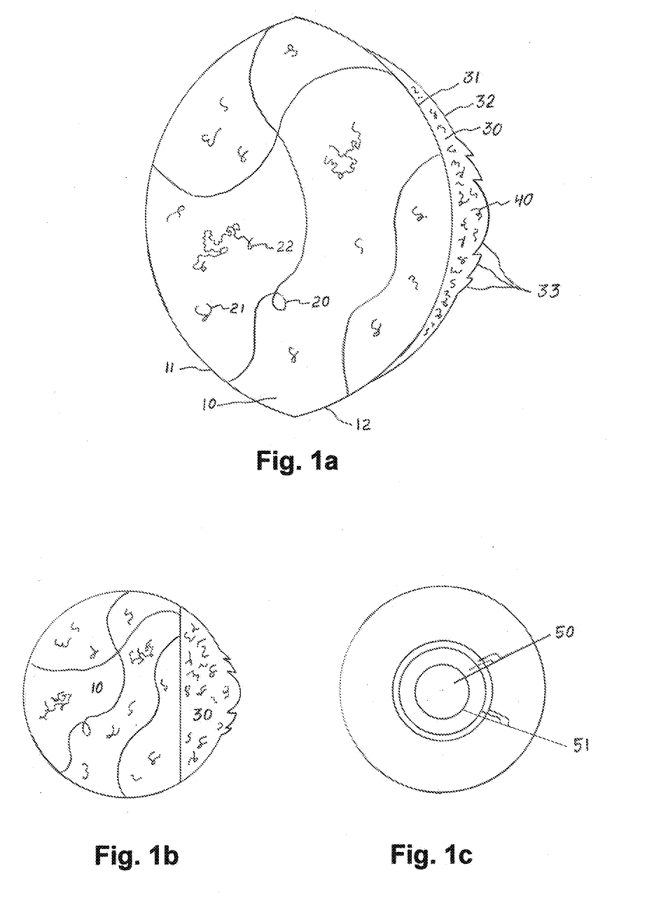

A light adjustable diffraction multifocal IOL, according to the present invention, is a mutifocal IOL whose optical power (prescription) may be adjusted after implantation and subsequent wound healing, wherein its multifocality is generated by a diffraction profile. An example of such a lens is illustrated in FIG. 1a where we see that it comprises a lens body 10 affixed to a diffraction element 30. The lens body is designed to function as a light adjustable optical element to provide far vision, while the diffraction element is designed to provide multifocality.

### The Lens Body

Referring to FIG. 1a, the lens body 10 is defined by an anterior surface 11 and a posterior surface 12 and, in general, it comprises (i) a first polymer matrix 20 and (ii) a refraction modulating composition (RMC) 21 dispersed therein.

The first polymer matrix 20, together with the anterior surface 11 and the posterior surface 12, form the optical element framework of the lens body which is generally responsible for many of the lens' optical properties. The first polymer matrix 20 must form a structure loose enough to allow the RMC components to freely diffuse throughout said first polymer matrix. It is also preferable that the first polymer matrix possess a relatively low glass transition temperature so that the IOL will exhibit elastomeric behavior.

The refraction modulating composition (RMC) 21 is capable of stimulus-induced polymerization, preferably photo-polymerization, and it is this mechanism that allows for post-surgical modification of the optical properties of the lens body 10. In particular, upon exposure to an appropriate energy source, the RMC is polymerized thereby forming the RMC matrix 22 in the exposed region of the lens body. The formation of the RMC matrix 22 typically increases the refractive index of the affected portion of the lens body. Moreover, after exposure, the RMC components in the unexposed region will migrate into the exposed region over time causing the lens to swell in the exposed region altering the shape of the lens body surface 10. Taken together, the changes in the refractive index and the shape of the lens body will alter the optical power (prescription) of the IOL thereby providing an IOL whose power may be adjusted after implantation and subsequent wound healing.

### The Diffraction Element

Still referring to FIG. 1a, we see that the diffraction element 30 is defined by an anterior surface 31 and a posterior surface 32 and, in general, it comprises a second polymer matrix 40 which forms the body of the diffraction element. This second polymer matrix 40 must form a structure tight enough to prevent the RMC components from diffusing into said second polymer matrix. It is also preferable that this second polymer matrix possess a relatively low glass transition temperature so that the IOL will exhibit elastomeric behavior.

FIG. 1a also shows a diffraction profile 33 forming part of the posterior surface 32 which includes the diffraction profile 33. It is this diffraction profile that provides the multifocality of the IOL. Because of the exacting tolerances necessary to effect the functionality of a diffraction profile, it is necessary that the physical structure of the diffraction element 30 not be modified by any post-surgical polymerization of the RMC components dispersed throughout the lens body 10. This is the reason that the second polymer matrix 40, dispersed throughout, and forming the framework of, the diffraction element 30, must form a structure tight enough to prevent the RMC components from diffusing into said second polymer matrix. In the alternative, a barrier material that is impervious to the RMC components may be located between the lens body 10 and the diffraction element 30 thereby preventing the RMC components from diffusing from said lens body into said diffraction element.

It is to be understood, that the interface between the lens body 10 and the diffraction element 30 make take any form; spherical, aspherical, concave, convex, or plano. An embodiment wherein the interface between the lens body 10 and the diffraction element 30 takes on a plano configuration is shown in FIG. 1b. It is also to be understood, that the diffraction element need not necessarily be multifocal in nature. For example, a diffraction element may be designed as a single vision lens used to correct for chromatic aberration in the lens element.

FIG. 1c shows a plan view of the the diffraction profile 33 which forms part of the posterior surface 32. In general, the diffraction profile 33 defines a series of concentric annular zones 50 of equal area. Adjacent annular zones are separated by optical steps of predetermined step heights. Each zone is bounded by a radius 51. For equal area zones, the outer boundary radius of each zone is given by the formula rₘ=mr₁², where rₘ is the radial distance from the center of the lens to the outer boundary of the mth annular zone.

The design of diffraction profiles for IOLs are more fully discussed in U.S. Pat. Nos. 4,210,391; 4,338,005; 4,340,283; 4,881,804; 4,881,805; 4,995,714; 4,995,715;, 4,881,804; 4,881,805; 5,017,000; 5,054,905; 5,056,908; 5,120,120; 5,121,979; 5,121,980; 5,144,483; 5,117,306 and 8,678,583 (Cohen); U.S. Pat. Nos. 5,076,684; 5,116,111 and 7,572,007 (Simpson, et al.); U.S. Pat. No. 5,129,718 (Futhey, et al.); and U.S. Pat. Nos. 4,637,697; 4,641,934 and 4,655,565 (Freeman)—the entire contents of which are incorporated herein by reference.

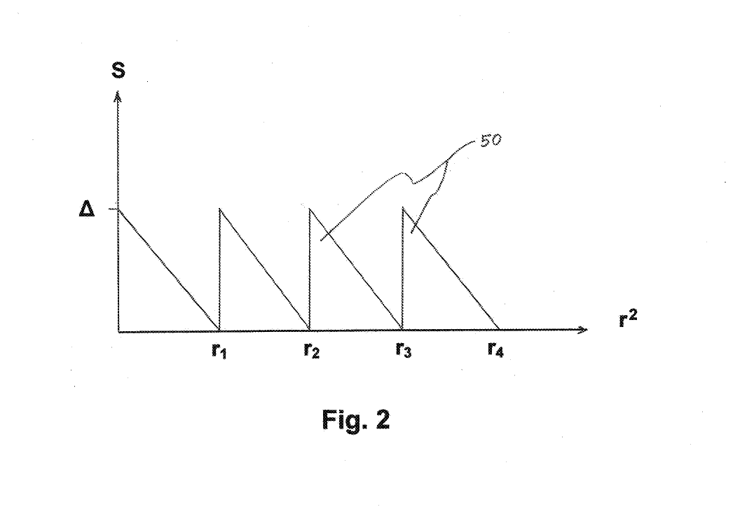

One embodiment of the diffraction profile 33 of FIG. 1c, is shown schematically in FIG. 2 with the vertical axis representing the optical step height S and the horizontal axis representing r², where r is the radial distance from the center of the IOL. We can see that in this embodiment, all of the zones 50 have the same step height Δ. However, It is understood that there are many other useful diffraction profile designs that can be incorporated within the scope of the present invention.

### The Polymers

As shown in FIG. 1a, the first polymer matrix 20 is dispersed throughout the lens body 10 and the refraction modulating composition (RMC) 21 is dispersed throughout said first polymer matrix 20, while the second polymer matrix 40 is dispersed throughout the diffraction element 30.

The materials used to form the first polymer matrix 20 and the refraction modulating composition (RMC) 21, of light adjustable IOLs are more fully discussed in U.S. Pat. Nos. 6,450,642; 6,813,097; 6,824,266 and 6,851,804 (Jethmalani, et al.); U.S. Pat. Nos. 6,749,632; 7,281,795 and 7,988,285 (Sandstedt et al.); U.S. Pat. No. 8,604,098 (Boydston, et al.)—the entire contents of which are incorporated herein by reference. Typical materials used to form the second polymer matrix 40 are more fully discussed in U.S. Pat. Nos. 2,976,576 and 3,220,960 (Wichterle, et al.); U.S. Pat. No. 4,440,918 (Rice, et al.) and U.S. Pat. No. 4,666,640 (Neefe)—the entire contents of which are incorporated herein by reference.

As disclosed in U.S. Pat. Nos. 6,450,642; 6,813,097; 6,824,266 and 6,851,804 (Jethmalani, et al.), in general, the first polymer matrix 20, of the lens body, and the refraction modulating composition (RMC) 21 dispersed therein, are selected such that the components that comprise the RMC are capable of diffusion within the first polymer matrix. In other words, a loose first polymer matrix should be paired with larger RMC components and a tight first polymer matrix should be paired with smaller RMC components. Additionally, for the purposes of the present invention, the second polymer matrix 40, of the diffraction element, should be chosen to form a matrix structure tight enough to prevent the RMC components from freely diffusing into said second polymer matrix 40.

### Method of Manufacture

Several methods of cast molding IOL lenses are known in the prior art, including injection molding, liquid injection molding, compression molding, and transfer molding. These methods generally involve introducing an uncured polymer into a mold cavity, in a predetermined amount and causing and/or allowing it to cure by use of various stimulus means. Some of these methods are more fully discussed in U.S. Pat. No. 5,620,720 (Glick, et al.), and U.S. Pat. No. 6,929,233 (Andin, et al.)—the entire contents of which are incorporated herein by reference.

It is also known in the prior art, to employ a spin casting technique to produce an IOL lens. Spin casting consists of polymerizing a suitable monomer composition in an open mold cavity with a concavely curved bottom surface that is rotating about an upright axis transverse to that surface. The effect of centrifugal and gravitational forces on the monomer composition determine the final lens power and shape. Once equilibrium is reached and said shape is obtained, the monomer is cured by use of various stimulus means. Spin casting methods are more fully discussed in U.S. Pat. Nos. 2,976,576 and 3,220,960 (Wichterle, et al.); U.S. Pat. No. 4,637,791 (Neefe); U.S. Pat. No. 4,680,149 (Rawlings, et al.)—the entire contents of which are incorporated herein by reference.

It has been known in the prior art, to mold items in which sections thereof are of different polymer compositions. One known technique is to first inject one polymer into a mold and, after it is cured, inject another different polymer therein so that the two different polymer materials will be maintained bonded yet distinct, one from the other. In another known technique, the different polymers are simultaneously injected into the mold. These molding techniques are are more fully discussed in U.S. Pat. No. 3,950,483 (Spier); U.S. Pat. No. 4,369,152 (Gray et al.); U.S. Pat. No. 6,248,266 (Gartley et al.); U.S. Pat. Nos. 6,391,230 and 6,732,994 (Sarbadhikari—the entire contents of which are incorporated herein by reference.

Lenses of the present invention can be manufactured by the prior art molding techniques referenced above, including cast molding or a combination of spin casting and conventional molding.

### Prior Art: Refractive Index Change

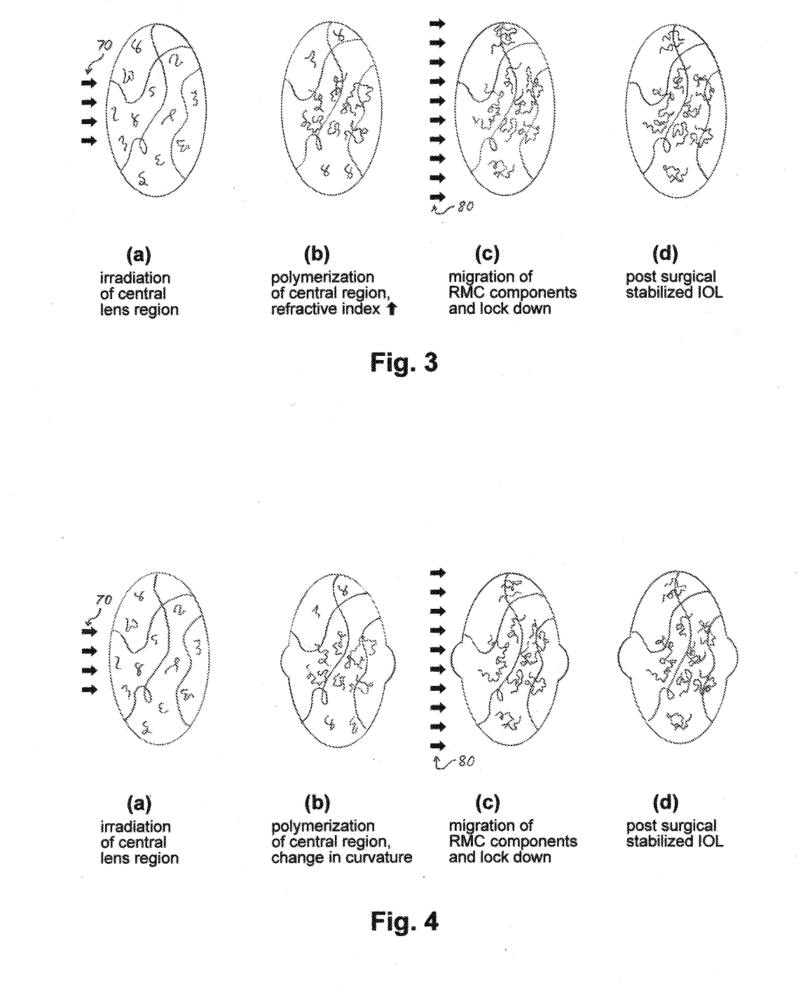

FIG. 3a-d illustrate a post-surgical procedure for inducing a power change in a prior art light adjustable IOL by altering the refractive index of said IOL. First, the central region of the IOL is exposed to an appropriate energy source 70 (FIG. 3a), whereby the refraction modulating composition (RMC), within said central region, polymerizes thereby forming the RMC matrix in the exposed region of the IOL (FIG. 3b). The RMC matrix causes an increase in the refractive index of this portion of the IOL. Additionally, after formation of the RMC matrix, the RMC components in the unexposed region will migrate into the exposed region.

If the central region is then re-exposed to the energy source 70, the RMC that has since migrated into the central region polymerizes to further increase the formation of RMC matrix. This process may be repeated until the central region of the optical element has reached the desired refractive index and therefore the desired power change. At this point, the entire IOL is exposed to the energy source 80 (FIG. 3c) to “lock-in” the desired lens property by polymerizing the remaining RMC components that are outside the exposed region thereby preventing any more migration of RMC components (FIG. 3d).

### Prior Art: Radius of Curvature Change

FIG. 4 illustrates a post-surgical procedure for inducing a power change in a prior art light adjustable IOL by altering the radius of curvature of said IOL. First, the central region of the IOL is exposed to an appropriate energy source 70 (FIG. 4a), whereby the refraction modulating composition (RMC), within said central region, polymerizes thereby forming an RMC matrix in the exposed region of the IOL.

After formation of the RMC matrix, the RMC components in the unexposed region will migrate into the exposed central region causing the lens to swell in the exposed central region. This leads to changes in the radius of curvature of the lens in either one or both of the anterior and posterior surfaces resulting in a change in the power of the IOL (FIG. 4b).

If the central region is then re-exposed to the energy source 70, the RMC that has since migrated into the central region polymerizes to further increase the formation the RMC matrix. This results in additional migration of RMC components and additional swelling. This process may be repeated until the central region of the optical element has reached the desired radius of curvature and therefore the desired power change. At this point, the entire IOL is exposed to the energy source 80 (FIG. 4c) to “lock-in” the desired lens property by polymerizing the remaining RMC components that are outside the exposed region thereby preventing any more migration of RMC components (FIG. 4d).

### Prior Art: Multifocality

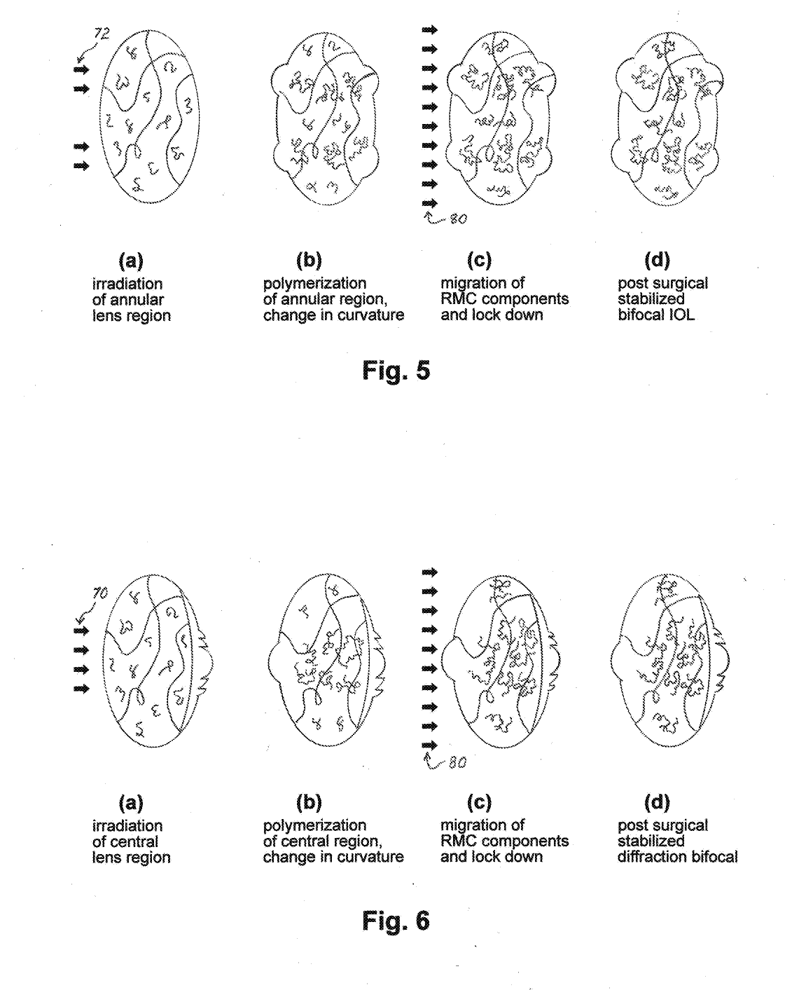

FIG. 5 illustrates a post-surgical procedure for creating a bifocal IOL by selectively altering the radius of curvature of the originally implanted single vision light adjustable IOL. First, an annular region of the IOL is exposed to an appropriate energy source 72 (FIG. 5a), whereby the refraction modulating composition (RMC), within said annular region, polymerizes thereby forming an RMC matrix in the exposed region of the IOL.

After formation of the RMC matrix, the RMC components in the unexposed region will migrate into the exposed annular region causing the lens to swell in the annular region. This leads to changes in the radius of curvature of the lens in the annular region thereby creating an annular ring with a power different from the power of central and peripheral regions of the IOL (FIG. 5b).

The application of the radiation source 72 to the annular ring may be repeated until a desired amount of power change has occurred in the annular region. After the lens has the desired optical characteristics, the lens is “locked-in” by irradiating the entire surface of the lens polymerizing the remaining RMC components (FIG. 5c) thereby producing a stabilized refractive bifocal IOL (FIG. 5d).

### Power Adjustment of Light Adjustable Diffraction IOL

FIG. 6 illustrates one possible post-surgical procedure for inducing a power change in an IOL of the present invention by altering the refractive index and radius of curvature of said IOL. First, the central region of the IOL is exposed to an appropriate energy source 70 (FIG. 6a), whereby the refraction modulating composition (RMC), within said central region, polymerizes thereby forming an RMC matrix in the exposed central region of the IOL. The RMC matrix causes an increase in the refractive index of this portion of the IOL. In Addition, after formation of the RMC matrix, the RMC components in the unexposed region will migrate into the exposed central region of the lens body 10 causing a swelling of said exposed central region of the lens body 10 (FIG. 6b).

There will, however, in the case of the lens of the present invention, be no swelling in the diffraction element which will thereby retain its structural integrity leaving the diffraction profile 33 undistorted. This is so, because the RMC components in the unexposed region cannot migrate into the refractive element 30 because of the tight nature of the polymer matrix 40 dispersed throughout said refractive element 30.

If the central region is then re-exposed to the energy source 70, the RMC that has since migrated into the region polymerizes to further increase the formation of the RMC matrix. This results in additional migration of RMC components and additional swelling. This process may be repeated until the exposed central region of the optical element has reached the desired radius of curvature and therefore the desired power change. At this point, the entire IOL is exposed to the energy source 80 (FIG. 6c) to “lock-in” the desired lens property by polymerizing the remaining RMC components that are outside the exposed region thereby preventing any more migration of RMC components (FIG. 6d).

Heretofore, the multifocality of light adjustable multifocal IOLs, was always achieved by refractive means. This was because such light adjustable multifocal IOLs were designed as a single uniform lens, wherein the entire lens would be deformed during post-surgical power adjustments. With the present invention using a composite lens structure, we can incorporate a diffraction element that is not deformed during post-surgical power adjustments.

Providing multifocality by incorporating a diffraction means allows for better resultant patient vision.

It is to be understood that there are many other useful lens designs, other than those specifically disclosed, that can be incorporated within the scope of the present invention

1. An intraocular lens, for surgical implantation into a human eye, comprising:
a. a lens body that includes a material that is optically reactive to an external stimulus, wherein the post-surgical application of said external stimulus induces a desirable modification of the optical properties of said lens body, and
b. a diffraction element that is optically unaffected by said external stimulus.

2. The intraocular lens of claim 1,
wherein the diffraction element is a multifocal diffraction element.

3. An intraocular lens comprising:
a. a lens body that comprises
i. a first polymer matrix, and
ii. a refraction modulating composition dispersed therein, and
b. a diffraction element comprising a second polymer matrix.

4. The intraocular lens of claim 3,
wherein the first polymer matrix functions as an optical lens; and
wherein the refraction modulating composition is capable of stimulus-induced polymerization, such that said stimulus-induced polymerization causes a desired change of power and or other optical properties of said optical lens; and
wherein the diffraction element comprises a multifocal diffraction profile that manifests two or more optical powers in said intraocular lens, and said multifocal diffraction element is relatively unaffected by the stimulus-induced polymerization of said refraction modulating composition.

5. The intraocular lens of claim 4,
wherein the refraction modulating composition is capable of polymerization by exposure to a laser light source such that said laser-induced polymerization causes a desired change of power and or other optical properties of said optical lens.

6. The intraocular lens of claim 4,
wherein the components of the refraction modulating composition are freely diffusible within the first polymer matrix within the lens body, but are not freely diffusible within the second polymer matrix within the diffraction element; and
wherein the first polymer matrix within the lens body and the second polymer matrix within the diffraction element possess a relatively low glass transition temperature such that the multifocal intraocular lens will exhibit elastomeric behavior.

7. The intraocular lens of claim 6,
wherein the refraction modulating composition is capable of polymerization by exposure to a laser light source such that said laser-induced polymerization causes a desired change of power and or other optical properties of said optical lens.
